(19) United States
(12) Patent Application Publication
(10) Pub. No.: US 2006/0286590 A1
(43) Pub. Date: Dec. 21, 2006

(54) Cis-regulatory modules

(75) Inventor: Michael J. Campbell, San Francisco, CA (US)

Correspondence Address: MIN, HSIEH & HACK LLP, 8270 GREENSBORO DRIVE, SUITE 630, MCLEAN, VA, 22102, US

(73) Assignee: Applera Corporation.

(21) Appl. No.: 11/455,668

(22) Filed: Jun. 20, 2006

Related U.S. Application Data
(60) Provisional application No. 60/692,187, filed on Jun. 20, 2005.

(51) Int. Cl. C12Q 1/68; G06F 19/00

(52) U.S. Cl. 435/6; 702/20

(57) ABSTRACT

A process for identifying a cis-regulatory module including aligning a target sequence with at least one sequence from a moderately distant species; determining a non-coding region of the target sequence, wherein the non-coding region comprises at least one of a high level of conservation and a suppression of indels; and identifying at least one cis-regulatory module in the target sequence is disclosed. Also disclosed is a method for providing assays to a consumer.

### CROSS-REFERENCE TO RELATED APPLICATIONS

This application claims priority to U.S. Provisional Patent Application Ser. No. 60/692,187, filed on Jun. 20, 2005, the disclosure of which is incorporated by reference in its entirety.

### FIELD OF THE DISCLOSURE

The present disclosure relates to at least one cis-regulatory module comprising at least one transcription factor binding site.

### BACKGROUND OF THE DISCLOSURE

Cis-regulatory modules (CRMs), usually about 300 to about 800 base pairs in length, can comprise transcription factor binding sites (TFBSs) and a sequence intervening between these sites. Currently, an algorithm can be run that searches for TFBSs on the DNA in and around a gene. However, because most TFBSs are short sequences of about 4 to about 8 base pairs in length many false positive signals are detected in the approximately 50,000 base pairs around the gene.

The DNA of functional CRMs displays extensive sequence conservation in comparisons of genomes from modestly distant species. Patches of sequence several hundred base pairs in length within these modules are often seen to be highly conserved with less insertions or deletions of sequence than seen in adjacent non-coding sequence, while the flanking sequence can often not be aligned. In the general case where the transcription factor binding sites are not known in advance, interspecific sequence comparison can be a method for physically identifying putative cis-regulatory modules in the intronic or intergenic DNA sequence of given animal genes. As has long seemed reasonable to assume, on the grounds that they are functionally essential, these key regulatory units of the genome can be evolutionarily conserved relative to a flanking sequence. Thus cis-regulatory modules can be detected computationally by interspecific comparison of the sequence surrounding the gene of interest, recognized as a block of sequence which has remained relatively similar between, for example, two species, excised by PCR and incorporated in an expression vector, and their function then studied by direct gene transfer methods.

The appropriate evolutionary species distance must be chosen; that is, not so close that unselected (i.e., “background”) sequence has not had time to diverge, but not so far that the pattern of conservation has been lost by too much divergence. But at the “right” distance, cis-regulatory modules stand out from the immediately flanking background as patches of well conserved sequence, usually several hundred base pairs in length, terminated at their boundaries by abrupt transitions to sequence that has diverged too greatly for easy computational alignment.

The use of interspecific sequence comparison to locate CRMs can decrease the percentage of false positives associated with using known algorithms to find TFBSs.

### SUMMARY OF THE DISCLOSURE

In accordance with the disclosure, there is provided a process for identifying a cis-regulatory module comprising aligning a target sequence with at least one sequence from a moderately distant species; determining a non-coding region of the target sequence, wherein the non-coding region comprises at least one of a high level of conservation and a suppression of indels; and identifying at least one cis-regulatory module in the target sequence.

In an embodiment, there is provided a process for identifying a transcription factor binding site comprising aligning a target sequence with at least one sequence from a moderately distant species; determining a non-coding region of the target sequence, wherein the non-coding region comprises at least one of a high level of conservation and suppression of indels; identifying at least one cis-regulatory module in the target sequence; and performing an algorithm to locate at least one transcription factor binding site within the at least one cis-regulatory module.

In another embodiment, there is provided a process for identifying a gene that is regulated by a transcription factor binding site comprising providing a first gene that encodes a first protein; and identifying sequences that the first protein is known to bind to within a cis-regulatory module near a second gene.

In yet another embodiment, there is provided a method for providing assays to a consumer comprising providing at least one web-based user interface selected from the group consisting of a) an interface configured to receive an order for at least one stock assay, b) an interface configured to receive a request for design of at least one custom assay and an order for said custom assay, and c) an interface configured for a consumer to perform at least one search for at least one information item chosen from cis-regulatory modules, transcription factor binding sites, genes whose proteins bind these CRM-embedded transcription factor binding sites, and SNPs that occur in CRM-embedded TFBs; and delivering to the consumer at least one assay chosen from a custom assay and a stock assay in response to the order.

Additional objects and advantages of the disclosure will be set forth in part in the description which follows, and can be learned by practice of the disclosure. The objects and advantages of the disclosure will be realized and attained by means of the elements and combinations particularly pointed out in the appended claims.

It is to be understood that both the foregoing general description and the following detailed description are exemplary and explanatory only and are not restrictive of the disclosure, as claimed.

The accompanying drawings, which are incorporated in and constitute a part of this specification, illustrate one (several) embodiment(s) of the disclosure and together with the description, serve to explain the principles of the disclosure.

### BRIEF DESCRIPTION OF THE DRAWINGS

FIG. 1 is a block diagram representing one method of providing assays to a consumer according to various embodiments.

FIG. 2 is a block diagram representing various configurations of a computer system of the present disclosure that can be used for distributing biotechnology products to a consumer.

FIG. 3 is a flow chart representative of various method configurations of the present disclosure that can perform by computing system configurations.

FIG. 4 is a flow chart illustrating the manner in which a user collects information for gene expression stock assays according to an embodiment.

FIG. 5 is a flow chart illustrating the order in which a user can perform a search for gene expression assays according to an embodiment.

FIG. 6 is a flow chart illustrating the manner in which a user can conduct a classification search for gene expression products performed according to an embodiment.

### DESCRIPTION OF THE EMBODIMENTS

Reference will now be made in detail to the present embodiments of the disclosure, examples of which are illustrated in the accompanying drawings. Wherever possible, the same reference numbers will be used throughout the drawings to refer to the same or like parts.

### Definitions

Allele. One of several alternative forms of a gene or DNA sequence at a specific chromosomal location (locus). At each autosomal locus an individual possesses two alleles, one inherited from the father and one from the mother.

Allele-specific Oligonucleotide (ASO). A synthetic oligonucleotide, often about 20 bases long, which hybridizes to a specific target sequence and whose hybridization can be disrupted by a single base pair mismatch under carefully controlled conditions. ASOs can often be labeled and used as allele-specific hybridization probes. They can also be designed to act as allele-specific primers in certain PCR applications.

Allelic association. Any significant association between specific alleles at two or more neighboring loci.

Alternative splicing. The natural usage of different sets of exons, to produce more than one product from a single gene.

Assay any of a number of nucleic acid assay systems (see U.S. Pat. No. 6,174,670, 2001). In various embodiments, an assay can comprise nucleobase polymers, such, as, for example, oligonucleotides, which constitute one or more probes and/or a forward and reverse primer. The assays can be configured to detect the presence of a SNP, the expression of a gene or the expression level of a gene. When using a TaqMan® procedure, the assay includes a TaqMan® probe, a forward primer and a reverse primer. See also “custom assay” and “stock assay.”

Alu repeat (or sequence). One of a family of about 750,000 interspersed sequences in the human genome that are thought to have originated from the 7SL RNA gene.

Amplicon. A region defined by pairing of forward and reverse primers around a target site.

Anticodon. A sequence of three consecutive bases in a tRNA molecule that specifically binds to a complementary codon sequence in mRNA.

Autocalling. The use of an automated system to make a determination of genotype.

Bioinformatics. The collection, organization and analysis of large amounts of biological data, using networks of computers and databases.

BLAST. Basic Local Alignment Search Tool—Algorithms for sequence searching. A fast technique for detecting subsequences that match given query sequence. BLAST is a heuristic search algorithm employed by computer programs to ascribe significance to sequence findings using well-known statistical methods, for example, a fast search algorithm to search DNA databases based upon sequence similarities. (See, for example, Altschul et al., J Mol Biol 215:403-10, 1990; Karlin et al., Proc. Nat'l Acad. Sci. USA 87: 2264-2268, 1990; Karlin et al., Proc. Nat'l Acad. Sci. USA 90: 5873-5877 1993; and Altschul et al., Nat. Genet. 6: 119-129 1994.) A BLAST analysis, in this context, refers to comparing sequences using a BLAST program such as blastp, blastn, blastx, tblastn, tblastx (accessible on the internet at http://www.ncbi.nim.nih.gov/BLAST/) or MPBLAST (Korf et al., Bioinformatics 16: 1052-1053 (2000). “BLASTING,” in this context, refers to comparing a sequence to sequences in a database, and identifying sequences contained in the database that are similar or identical to the sequence or its complement.

BLASTn. Search of a DNA sequence against a DNA sequence database.

Calling. The process of determining a genotype.

cDNA. Complementary DNA—a single stranded DNA sequence that was generated from and complementary to an mRNA sequence by reverse transcription. cDNA sequences contain only genes that code for protein (no non-coding DNA is included).

cDNA Library. A collection of single stranded DNA sequences that represent DNA that is translated into protein. cDNA libraries are generated from mRNA. They are designed to represent the portion of the genome that is present as mRNA in a given cell on its way to synthesizing the proteins represented in that cell.

Centimorgan (cM). A unit of measure of recombination frequency. One centimorgan is equal to a 1% chance that a marker at one genetic locus will be separated from a marker at a second locus due to crossing over in a single generation. In human beings, 1 centimorgan is equivalent, on average, to 1 million base pairs.

Common SNPs. SNPs which have a minor allele frequency equal to or greater than a minimum percent of occurrence in an overall population, e.g. a population of humans or, in certain subsets of the overall population. Such subsets can include an ethnically defined subset population. This can be assessed using samples from mixed populations or from specific populations such as Caucasian populations or African American populations as are available from repositories such as, for example, the Coriell Cell Repositories (Coriell Institute for Medical Research, Camden, N.J.).

Conserved sequence. A base sequence in a DNA molecule (or an amino acid sequence in a protein) that has remained essentially unchanged throughout evolution.

Consumer. Encompasses customers and other users of the products and services provided in configurations of the present disclosure. Unless explicitly stated otherwise, it is permitted but not required that configurations of the present disclosure precondition distribution on receipt of a payment or a promise to pay from the consumer for the distributed products or services. The terms “consumer,” “requester,” “user” and “investigator” refer to entities different from the supplier and distributor. The terms “consumer,” “requester,” “user” and “investigator” are often used interchangeably herein. However, in any given situation, it is possible that the consumer, the requester, the user and/or the investigator are different entities or individuals, which themselves may (or may not) be related by agency. For example, the consumer, requester, user and investigator in one instance may be a single individual engaged in research, such as at a college or university. As another example, the consumer may be a medical institution, the investigator may be a physician or researcher employed by the medical institution, and the requestor may be an assistant of the investigator. Also herein, the term “user” is frequently used to refer to an entity (such as a consumer, a requester, or an investigator) who can access a computer system.

Contig display name. The contig display name is the genome assembly (GA) name as used in some configurations of gene exploration systems.

Cryptic splice site. A sequence that resembles an authentic splice junction site and which can, under certain circumstances, participate in an RNA splicing reaction.

Custom assay. An assay that is designed from specifications that are generally related to the target sequence, but that do not contain information on the specific sequence of the probe or probes and primers.

dbSNP rs#ID. A specific field for searching for a SNP according to a dbSNP reference cluster ID.

dbSNP ss#ID. A specific field for searching for a SNP according to a dbSNP assay ID.

Deletions can be generated by removal of a sequence of DNA, such as at least one nucleotide base, the regions on either side being joined together.

Discriminator. A procedure in which the “A-statistic” is used to screen out assemblies that are likely to be stacked regions of repetitive sequence that can be from more than one area of the genome.

Distribute. As used herein, the terms “distribute” and “provide” may be used synonymously, and are intended to encompass selling, marketing, or otherwise providing a product or service.

Distributor. As used herein the terms “distributor,” “provider” and “supplier” are used to refer to an entity or entities that distributes and/or supplies products and/or services. The terms “distributor,” “provider,” and “supplier” can encompass sellers, marketers, and other providers of such products and services. The distributor, supplier, and provider can refer to the same entity, to two different entities, or to three different entities. In the description herein, it may be generally assumed that the manufacturer can be the supplier and distributor of the assay-related products and services described herein. However, in some configurations of the present disclosure, the distribution of the assay-related products and services described herein may be performed by an entity other than the manufacturer who supplies them.

DNA sequence. The relative order of base pairs, whether in a fragment of DNA, a gene, a chromosome, or an entire genome. See base sequence analysis.

Domain. A discrete portion of a protein with its own function and structure. The combination of domains in a single protein determines its overall function. The domain of a chromosome can refer either to a discrete structural entity defined as a region within which a supercoiling can be independent of other domains; or to an extensive region including an expressed gene that can have a heightened sensitivity to degradation by the enzyme DNAase I.

ENTREZ. NCBI's (National Center for Biotechnology Information) search and retrieval system for their data sets. It organizes GenBank sequences and links them to the literature sources in which they originally appeared.

EST. Expressed Sequence Tag. A sampling of sequence from a cDNA library. A short sequence of a cDNA clone for which a PCR assay is available.

Euchromatin. The fraction of the nuclear genome that contains transcriptionally active DNA and which, unlike heterochromatin, adopts a relatively extended conformation.

Exon(s). The protein-coding sequences of genes. Exons only comprise about 10% of the human genome. A segment of a gene that is decoded to give a mRNA product or a mature RNA product. Individual exons may contain coding DNA and/or noncoding DNA (untranslated sequences). See introns.

FASTA (file or format). A DNA sequence format that begins with a single line of text description that is less than 80 characters in length, followed by the DNA sequence file.

FASTA Search. A database search tool used to compare a nucleotide or peptide sequence to a sequence database. The program is based on the rapid sequence algorithm described by Lipman and Pearson.

Fragments. Small sections of DNA.

Frameshift mutation. A mutation that alters the normal translational reading frame of a DNA sequence.

GenBank. The public DNA sequence database maintained by the National Center for Biotechnology Information (NCBI), part of the National Library of Medicine.

Gene Exploration Platform (also referred to as Gene Exploration System). A web-based user interface configured to provide searchable information related to one or more genomes and/or transcriptomes and/or proteomes.

Gene families. Groups of closely related genes that make similar products.

Gene Ontology (GO). A controlled vocabulary for the description of the molecular function, biological process and cellular component of gene products which can be applied to all eukaryotes. The GO terms can be used as search identifiers.

Gene prediction. The process of using computational methods that search for known indicators of coding regions in the raw genomic sequence. These indicators include codon use bias, lack of stop codons, similarity of the translated protein sequence to known proteins, upstream regulators, splice sites, start codon. The outcome can be a set of exons that define a predicted gene.

Gene region. A linear stretch of genomic DNA which serves as a functional gene region comprising cis-acting regulatory regions, transcribed regions, and intervening sequences as well as 10 kilobase pairs of 5′ flanking sequence and 10 kilobase pairs of 3′ flanking sequence.

Genomics. The study of the genetic material of an organism; the sequencing and characterization of the genome and analysis of the relationship between gene activity and cell function. The genetic material includes exons, introns, regulatory sequences, repeat elements and all other unidentified regions of the genome.

GI. GenBank Identifier, a unique number assigned to protein and nucleotide sequences in the GenBank database.

GT-AG rule. Rule that describes the presence of these constant dinucleotides at the first two and last two positions of introns of nuclear genes.

Haplotype. A series of alleles found at linked loci on a single (paternal or maternal) chromosome.

Heterochromatin. A region of the genome, which remains highly condensed throughout the cell cycle and shows little or no evidence of active gene expression.

Homologies. Similarities in DNA or protein sequences between individuals of the same species or among different species. Homologous chromosomes: a pair of chromosomes containing the same linear gene sequences, each derived from one parent. Homologous chromosomes (homologs): two copies of the same type of chromosome found in a diploid cell, one having being inherited from the father and the other from the mother. Homologous genes (homologs): two or more genes whose sequences can be significantly related because of a close evolutionary relationship, either between species (orthologs) or within a species (paralogs).

HSPs. High-scoring Segment Pairs; two sequence fragments of arbitrary but equal length with an alignment that can be locally maximal and for which the alignment score meets or exceeds a threshold (cutoff) score. These can be generated by BLAST.

Informatics. The study of the application of computer and statistical techniques to the management of information. In genome projects, informatics includes the development of methods to search databases quickly, to analyze DNA sequence information, and to predict protein sequence and structure from DNA sequence data.

Introns. DNA sequences in genes, which have no protein-coding function. Other non-coding regions include control or regulatory sequences and intergenic regions whose functions are unknown. Noncoding DNA separates neighboring exons eukaryote genes. During gene expression, introns, like exons, can be transcribed into RNA, but the transcribed intron sequences can be subsequently removed by RNA splicing and are not present in mRNA.

Investigator. See “consumer.”

Linkage map. A map of the relative positions of genetic loci on a chromosome, determined on the basis of how often the loci are inherited together. Distance is measured in centimorgans (cM).

Linker (or adaptor oligonucleotide). A double-stranded oligonucleotide that can be ligated to a cloned DNA of interest in order, for example, to facilitate its ability to be cloned.

Marker. An identifiable physical location on a chromosome (e.g., restriction enzyme cutting site, gene) whose inheritance can be monitored. Markers can be expressed regions of DNA (genes) or some segment of DNA with no known coding function but whose pattern of inheritance can be determined. See RFLP, restriction fragment length polymorphism.

Master cluster. A “super cluster” that can be formed by joining clusters and singletons that have representative clones with significant matches (a Product Score of 40 or more) to the same gene. The master cluster is named after the cluster (or singleton) with the highest Product Score.

Mate pairs. A pair of reads that are in opposite orientations and at a distance from each other approximately equal to the insert length.

Messenger RNA (mRNA). RNA that serves as a template for protein synthesis. See genetic code.

Missense mutation. A nucleotide substitution that results in an amino acid change.

mRNA (Messenger RNA). The nucleic acid intermediate that can be used to synthesize a protein. The mRNA corresponds to one strand of the DNA and the sequence of the mRNA can be identical to the sequence of the DNA, except for the replacement of a T (thymine) with U (uracil).

Mutation frequency. Is the frequency at which a particular mutant can be found in a population.

NCBI. The National Center for Biotechnology Information, which can be accessed at the web site http://www.ncbi.nim.nih.gov.

Nonsense mutation. A mutation that occurs within a codon and changes it to a stop codon.

Normalized Library. A cDNA library from which most of the highly expressed sequences have been removed in order to represent a greater proportion of low-abundance messenger RNAs. Normalized libraries are not an accurate reflection of a tissue's gene-expression profile.

Nucleobase. Any nitrogen-containing heterocyclic moiety capable of forming Watson-Crick hydrogen bonds in pairing with a complementary nucleobase or nucleobase analog, e.g. a purine, a 7-deazapurine, or a pyrimidine. The present disclosure in some configurations uses assays based upon probes that can be polynucleotides or polymeric forms of other nucleobases such as nucleic acid analogs. Typical nucleobases can be the naturally occurring nucleobases adenine, guanine, cytosine, uracil, thymine, and analogs (Seela, U.S. Pat. No. 5,446,139) of the naturally occurring nucleobases, e.g. 7-deazaadenine, 7-deazaguanine, 7-deaza-8-azaguanine, 7-deaza-8-azaadenine, inosine, nebularine, nitropyrrole (Bergstrom, (1995) J. Amer. Chem. Soc. 117:1201-09), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman (1989) in Practical Handbook of Biochemistry and Molecular Biology, pp. 385-394, CRC Press, Boca Raton, Fla.). Nucleobases that are nucleic acid analogs include peptide nucleic acids in which the sugar/phosphate backbone of DNA or RNA has been replaced with acyclic, achiral, and neutral polyamide linkages. The 2-aminoethylglycine polyamide linkage with nucleobases attached to the linkage through an amide bond has been reported (see, for example, Buchardt, WO 92/20702; Nielsen (1991) Science 254:1497-1500; Egholm (1993) Nature 365:566-68).

Open Reading Frame (ORF). A stretch of nucleotide sequence with an initiation codon at one end, a series of triplet codons and a termination codon at the other end: potentially capable of coding for an as yet unidentified peptide or protein.

Ortholog. One of a set of homologous genes in different species (e.g. SRY in humans and Sry in mice).

Panther. Celera Genomics's proprietary protein classification software that allows hierarchical classification of protein families and subfamilies to further aid in identifying probable protein function. Panther facilitates target identification and prioritization by allowing more accurate predictions of protein function.

Paralog. One of a set of homologous genes within a single species.

Pharmacogenomics. The study of the stratification of the pharmacological response to a drug by a population based on the genetic variation of that population.

Phrap. Developed by Phil Green at the University of Washington, “Phil's Revised Assembly Program” is a tool for assembling shot-gun sequenced DNA fragments.

PHYLIP. Program Package created by J. Felsenstein for Phylogenicity.

Physical map. A map of the locations of identifiable landmarks on DNA (e.g., restriction enzyme cutting sites, genes), regardless of inheritance. Distance can be measured in base pairs. The relative positions of regions can be determined by physical measurements, such as by electron microscopy, restriction analysis, or sequence determination. For the human genome, the lowest-resolution physical map is the banding patterns on the 24 different chromosomes; the highest-resolution map would be the complete nucleotide sequence of the chromosomes.

Point mutation. A mutation causing a small alteration in the DNA sequence at a locus, often a single nucleotide change.

Polygenic character. A character determined by the combined action of a number of genetic loci. Mathematical polygenic theory assumes there can be very many loci, each with a small effect.

Polygenic disorders. Genetic disorders resulting from the combined action of alleles of more than one gene (e.g., heart disease, diabetes, and some cancers). Although such disorders can be inherited, they depend on the simultaneous presence of several alleles; thus the hereditary patterns can be usually more complex than those of single-gene disorders.

Polymorphism. Difference in DNA sequence among individuals. Genetic variations occurring in more than 1% of a population would be considered useful polymorphisms for genetic linkage analysis.

Precomputes. A series of computational analyses of Celera Genomics data to public data. The analyses used include gene prediction (GRAIL, Genscan, FgenesH), BLAST computes using several public and proprietary datasets (nraa, CHGD, RefSeq) to show similarity, and polishing of the BLAST results to find consensus splice sites using SIM4 or Genewise with sequences that can be highly similar to the genomic sequence.

Primer. A primer comprises a polymer of nucleobases, such as, for example, an oligonucleotide, the sequence of which is complementary to a target sequence, or to the complement of a target sequence. In certain aspects, the 3′ end of an oligonucleotide primer can be extended by a DNA polymerase. The primer is short relative to the target nucleic acid. A primer sequence in some configurations comprises from about ten to about fifty nucleotides, and in some configurations comprises from about six, about eight, about ten, about thirteen up to about thirty nucleotides and any length there between. In most cases, PCR involves a forward primer and a reverse primer, which hybridize to opposite strands in a target sequence.

Probe. A “probe” comprises an oligonucleotide that hybridizes to a target sequence. In the TaqMan® assay procedure, the probe hybridizes to a portion of the target situated between the binding site of the two primers. A probe can further comprise a reporter group moiety. In some configurations, the reporter group moiety can be a fluorophore moiety. The reporter group can be covalently attached directly to the probe oligonucleotide, in some configurations to a base located at the probe's 5′ end or at the probe's 3′ end. The reporter group may also be attached to a minor groove binder (MGB), which can be itself covalently attached to the probe (Afonina et al., Nucleic Acids Research 25: 2657-2660 (1997); Kutyavin et.al., Nucleic Acids Research 28: 655-661 (2000)). The MGB is, in some configurations, attached to the 3′ end of the probe, either directly to the oligonucleotide or else to the fluorophore moiety or to the quencher moiety. A probe comprising a fluorophore moiety can also further comprise a quencher moiety. The quencher moiety is, in some configurations, a non-fluorescent quencher (NFQ). In some configurations, in probes designed for SNP detection, the fluorophore and the quencher can be attached to the oligonucleotide on opposites sides of the SNP nucleotide. A probe comprises about eight nucleotides, about ten nucleotides, about fifteen nucleotides, about twenty nucleotides, about thirty nucleotides, about forty nucleotides, or about fifty nucleotides. In some configurations, a probe comprises from about eight nucleotides to about fifteen nucleotides. As used herein, the use of the term “a probe” (singular) is intended to include or refer to two bi-allelic probes in the case of SNP assays, unless stated otherwise.

Proteome. The full set of proteins encoded by a genome.

Provide. See “Distribute.”

Provider. See “Distributor.”

Query. The DNA sequence used to search a database.

Radiation hybrid. A type of somatic cell hybrid in which fragments of chromosomes of one cell type can be generated by exposure to X-rays, and are subsequently allowed to integrate into the chromosomes of a second cell type.

Real time. The term “real time” is always spelled out in full. The abbreviation “RT,” as used herein, always refers to “reverse transcriptase.”

Receptor. A molecule (usually a protein) that spans a cell membrane, receives extracellular signals, and transmits them into the cell.

Regional overlay. Celera regional overlays can be created from Celera fragments and mate pair links, and external finished clones and unordered contigs from unfinished clones, which are referred to as BACs. The Celera Regional Assembler takes the external data and uses Celera fragments and mate pairs to order and orient the contigs within BACs, filling in gaps where possible.

Regulatory regions or sequences. A DNA base sequence that controls gene expression.

Repetitive DNA. A set of nonallelic DNA sequences which show considerable sequence homology.

Requestor. See “consumer.”

Reverse transcriptase (RT). The abbreviation “RT” is used herein exclusively as an abbreviation for “reverse transcriptase.” The term “real time” is always spelled out in full.

Scaffolds. Sets of contigs that can be ordered and oriented using enforcing mate pairs.

Sequence homology. A measure of the similarity in the sequence of two nucleic acids or two polypeptides.

Sequence tagged site (STS). Short (200 to 500 base pairs) DNA sequence that has a single occurrence in the human genome and whose location and base sequence are known. Detectable by polymerase chain reaction, STSs can be useful for localizing and orienting the mapping and sequence data reported from many different laboratories and serve as landmarks on the developing physical map of the human genome. Expressed sequence tags (ESTs) can be STSs derived from cDNAs.

Significant complementarity. Includes complementarity sufficient to interfere with the analysis of a target sequence. Significant complementarity can comprise, in non-limiting example, at least about 40% or greater sequence identity with the complement of a target sequence.

Single Nucleotide Polymorphism (SNP). Replacement, loss, or addition of one nucleotide (either A, C, G or T) in the DNA sequence. There are probably several million SNPs throughout the genome, and these alleles account for much of the variation seen in the human population. These predominately biallelic polymorphisms can exist in varying ratios in the population ranging from very rare alleles (1-5% frequency) to common alleles (20-50% frequency).

Splice acceptor site. The junction between the end of an intron terminating in the dinucleotide AG, and the start of the next exon.

Splice donor site. The junction between the end of an exon and the start of the downstream intron, commencing with the dinucleotide GT.

Stock assay. A pre-designed assay that does not require custom design. In some configurations of the present disclosure, an inventory of stock assays can be maintained from which users can place orders.

Stringency. A parameter for filtering the results of a query based on how closely related the sequences in a cluster must be.

Subject. A DNA sequence that produces a match in a blast search.

Supplier. See “Distributor.”

SWISSPROT. European annotated non-redundant protein sequence database; most highly annotated protein database.

TA. Transcript assembly. Celera assembly of public EST.

Tandem repeat sequences. Multiple copies of the same base sequence on a chromosome; used as a marker in physical mapping.

Target. A biological sample comprising a nucleic acid. A target can comprise a single-stranded or double-stranded nucleic acid, and can comprise a RNA or a DNA. A RNA can be, in non-limiting example, a messenger RNA (mRNA), a primary transcript, a viral RNA, or a ribosomal RNA. A DNA can be, in non-limiting example, a single-stranded DNA, a double-stranded DNA, a cDNA, a viral DNA, an extrachromosomal DNA, or a mitochondrial DNA. A skilled artisan will recognize from the context of usage whether a target nucleic acid is single-stranded or double-stranded.

TBLASTn. A BLAST search of a protein sequence against a nucleotide sequence database that has been translated in all six frames.

Trace Files. The product of sequencing completed by the ABI 3700 Prism. After going through stringent quality control processes, trace files can be then used as data input for assembly.

Transcriptome. The full complement of activated genes, mRNAs, or transcripts expressed from a genome.

TREMBL. Translated EMBL, a compilation of the EMBL DNA data library.

UniGene database. A public database, maintained by NCBI, which brings together sets of GenBank sequences that represent the transcription products of distinct genes.

Unique clone. A sequence that has no match in GenBank or other public databases.

Unique singleton. A clone that does not cluster and has no match in the public databases.

UTR (untranslated region). Noncoding region found at the 5′ or 3′ termini of mRNA.

Untranslated sequences. Noncoding sequences found at the 5′ and 3′ termini of mRNA.

User. See “consumer.”

The present disclosure relates to a process for identifying CRMs comprising TFBSs, wherein the CRMs can be located by using interspecific sequence comparison of several sequences, such as at least two sequences. In an embodiment, a stretch of DNA, such as about a 50 kb stretch, either singly or in separate smaller pieces, can be aligned with at least one sequence from a moderately distant species. One of ordinary skill in the art would be able to determine a moderately distant species based upon reviewing the evolution that occurred between genomes. For example, one of ordinary skill in the art could perform a BLASTn for several gene regions. If the region is too highly conserved, then there has not been enough evolution and the two species are too closely related. Similarly, if the region is not highly conserved, then there has been too much evolution and the two species are too distantly related. In an embodiment, a moderately distant species can be a sequence wherein more than or equal to about 10% of the nucleotides in a non-gene, non-repeat region have changed or been disrupted by insertions or deletions as compared to the target sequence. In another embodiment, a moderately distant species can be a sequence wherein there is alignment with the target sequence over at least about 30% of the nucleotides in a non-gene, non-repeat region.

Alignment of the sequences can reveal patches of sequences, such as a length of about 300-1500 bp region wherein there can be a statistically significant level of sequence conservation, and/or a suppression of indels (insertions/deletions) as, for example, compared to other non-coding regions. A single such stretch can be a CRM. A CRM can comprise one or many TFBSs, often with short stretches of sequence between the TFBSs. By identifying CRMs using this method, TFBSs are searched only within the CRM region, rather than across the initial 50 kb region. By substantially reducing the amount of DNA that needs to be searched for the TFBSs, the percentage of false positives can be reduced from, for example, about 1:20 being correct to about 1:2 or more being correct.

In an embodiment, the tracts of sequence from the genomic regions surrounding the relevant cis-regulatory modules in a species can be determined by using primers that lie outside of the highly conserved protein coding regions. (Sequences adjacent to CRMs should be known before CRM identification can occur. These adjacent sequences may not be aligned across species, but their sequence is known. We may want to axe this section?) In order to find suitable conserved regions for primer design, computer programs, such as BLASTn and Family Relations, can be used. In an embodiment, Family Relations, at a window size of 10 bp and a similarity of 100%, can reveal tracts of conserved sequence which can be easily seen in dot plots. These highly conserved regions can be used as likely primer targets. Primers can be designed using a computer program, such as Eprimer3. In an embodiment, primer pairs can be selected to yield overlapping products for sequencing.

In an embodiment, appropriate bacterial artificial chromosomes (BAC) inserts can serve as templates in standard PCR reactions. For sequencing reactions the amplified products can be gel-purified and the PCR primers can be used as sequencing primers in standard sequencing reactions, such as ABI Big Dye. Moreover, the sequencing reactions can be assembled with any known computer program, such as Phred-Phrap-Consed, and can be mapped onto any species sequence by, for example, Crossmatch. This map can then be viewed in another program, such as Family Relations.

An assembled species sequence can be preliminarily aligned to another species BAC sequences using, for example, BLASTn, in order to choose suitable regions for alignment with, for example, ClustalW. Regions marked by long indels can be examined by hand to confirm proper alignment. Indentities, single base pair substitutions, and number and size of gaps can be tabulated from for example, the ClustalW output. Primer walking methods, which are known to those of ordinary skill in the art, can be used to fill in any sequence gaps and to obtain additional sequence.

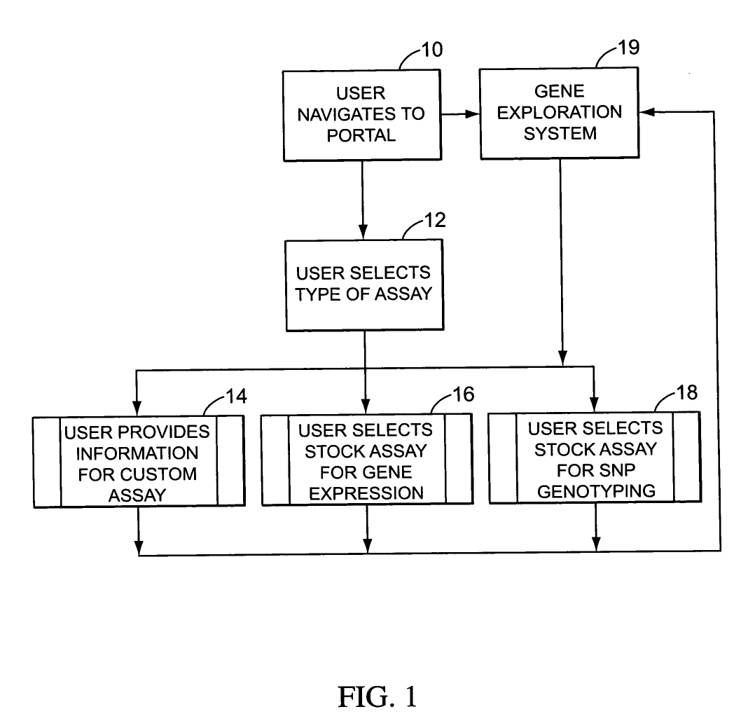

According to various aspects of the system disclosed herein, the user can use a web based portal to order products associated with conducting assays. The web based portal can be used to order custom assays and/or stock assays. In this regard, the user can initially navigate to the portal as shown in block 10 of FIG. 1, although it will be understood that any suitable portal may be used. Once the user arrives at the portal, the user can determine the type of assay that is desired as represented by block 12. For example, a user can desire to order a custom assay, a stock assay that can be used for gene expression experiments, or a stock assay that can be used for SNP genotyping experiments. It will be understood, however, that this set of assays is only exemplary in nature and can also include other assays and/or related products.

Depending upon the type of assay which the user desires, the processing can differ. For example, if the user desires to obtain a custom assay, the system can proceed to obtain from the user information which can be useful to deliver the custom assay to the user as indicated by block 14. Similarly, if the user desires to obtain an assay for gene expression experimentation, the system can proceed to obtain the information which can be useful to generate such an assay as represented by block 16. In addition, if the user desires to obtain an assay for SNP genotyping, the system can proceed to collect information useful to providing such an assay as represented by block 18. Further, the user can desire to use the gene exploration system as indicated by block 19.

Some configurations provide a gene exploration system or platform 19 that allows the user to perform in silico research which can assist the user in the process of assay selection. Gene exploration system 19 can be accessed directly from the portal 10 or from selection screens from custom assay and/or stock assay blocks 14, 16, and 18. For example, if a user has entered a custom or stock assay screen and wants to obtain further genomic information about a given assay, or if a user decides to perform further research prior to ordering a gene, an appropriate entry link to the gene expression system can be accessed.

Gene exploration system 19 can provide access to a set of genomic and biomedical data from public and/or private sources. Some configurations can provide integrated access to such data from Celera, GenBank, and other public and private data sources. Computational tools can also be provided to facilitate the viewing and analyzing of gene structure and function, genome structure and physical maps, and/or proteins classified by family, function, process, and/or cellular location. An intuitive user interface can be provided that organizes information for easy navigation and analysis.

In certain configurations, the gene exploration system 19 can provide the user with a link to a genome navigation page. Several options can be provided for genome navigation, including, for example, human, mouse, human and mouse comparative genomics, protein classification, and pharmacogenomics. For example, in some configurations, the genome navigation option can be configured to provide users with the capability to browse and search genome maps, genome assembly, and gene data.

A protein classification option can allow the user to browse and/or search at least one protein information database. Database capabilities can include, for example, browsing and text searching Celera PANTHER™ families and gene ontology classification data.

The pharmacogenomics option available in some configurations can provide the user with the ability to search against at least one SNP database, for example, the Celera Human SNP Reference Data database.

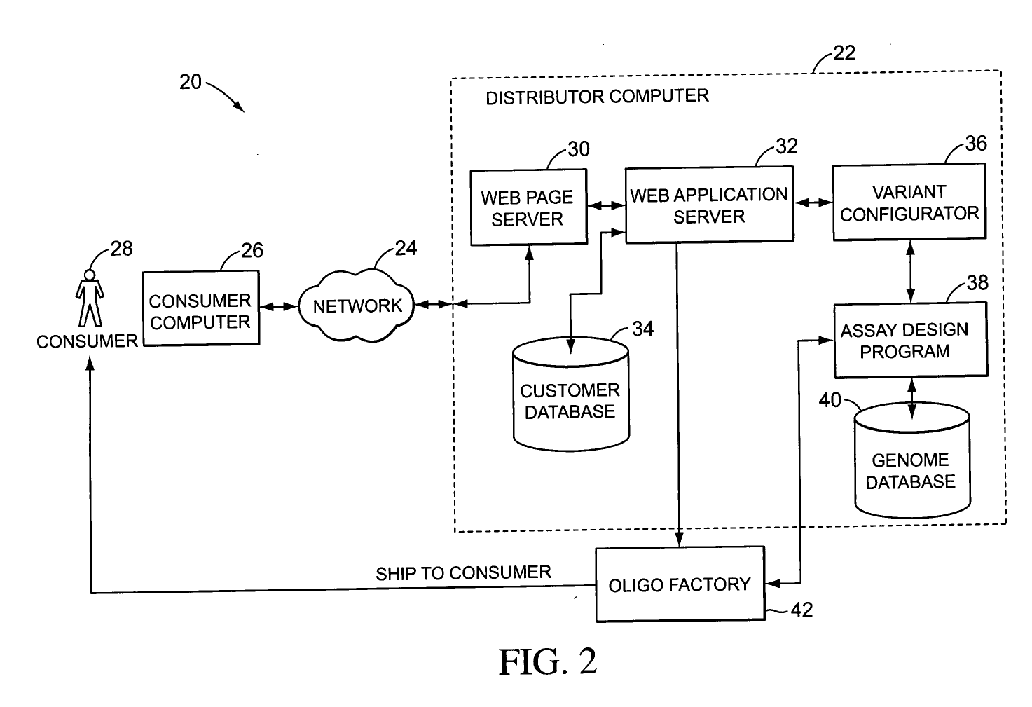

In various configurations of the present disclosure and referring to FIG. 2, a computing system 20 comprising a plurality of computers 22, 26 can be utilized to distribute information, products and services such as the custom assays and/or stock assays described herein, to a user or consumer 28. A first computer 22 (i.e., a distributor computer) on a computer network 24 (e.g., a public network, such as the Internet) can interact with a consumer 28 using a second computer 26 (i.e., a consumer computer) to obtain information that can be associated with a human or nonhuman target DNA (or RNA) sequence, which may include SNP and/or exon locations, i.e., the sequence itself, the SNP and/or exon locations themselves, or other information from which these items can be determined such as, for example, a gene name, accession number, etc. In some configurations, this interaction can be initiated by consumer 28 typing a uniform resource locator (URL) into a web browser running on consumer computer 26 and downloading a hypertext mark-up language (HTML) or other type of web page serving as a web portal (such as to which the user navigates in block 10 of FIG. 1) from a server 30 running on distributor computer 22.

The web page displayed on consumer computer 26 can include various types of introductory and sales information, provide a login for authorized user/purchasers, and solicit the DNA (or RNA) sequence and other information, as is necessary or desirable. In some configurations, the initial web page can be one of several web pages provided by server 30 that interact with consumer 28 to obtain information. For example, in some configurations, the initial web page accessed by consumer 28 can be a corporate web site that provides information for consumer 28 as well as a form in which consumer 28 types identifying information using consumer computer 26. Distributor computer 22 can receive the information entered by consumer 28 and sent by consumer computer 26 via computer network 24.

In some configurations, distributor computer 22 can verify the identity of consumer 28 and his or her qualifications to access a sales page and to purchase assays from the distributor. For example, this verification can be performed by a web application server 32 (for example, the IBM® WEBSPHERE® Application Server available from International Business Machines Corporation, Armonk N.Y.) running on distributor computer 22 with reference to a consumer database 34 of qualified consumers and consumer identifications. If consumer 28 cannot be verified or is not qualified to make a purchase, this information can be returned by web application server 32 and web page server 30 via computer network 24 to consumer 28, and consumer 28 will not be allowed to complete a purchase and/or to access additional information.

In some configurations, a variant configurator 36 (such as SELECTICA® Configurator™, available from Selectica, Inc., San Jose, Calif.) can interact with consumer 28 via network 24 to produce a list of specified characteristics. Configurator 36 can be an automated decision tree that produces the input for assay design program 38 and that ensures that input parameters to assay design program 38 are within bounds that can be handled by program 38. If there are no errors, assay design program 38 then uses a lookup process, a design process, or another suitable method to provide a forward primer sequence, a reverse primer sequence, and a probe sequence that have the specified characteristics.

An oligo factory 42 can manufacture at least one assay having components including a forward primer, a reverse primer and a probe and ships the manufactured assay to the consumer. The forward primer, reverse primer, and probe can be manufactured in accordance with a validated sequence.

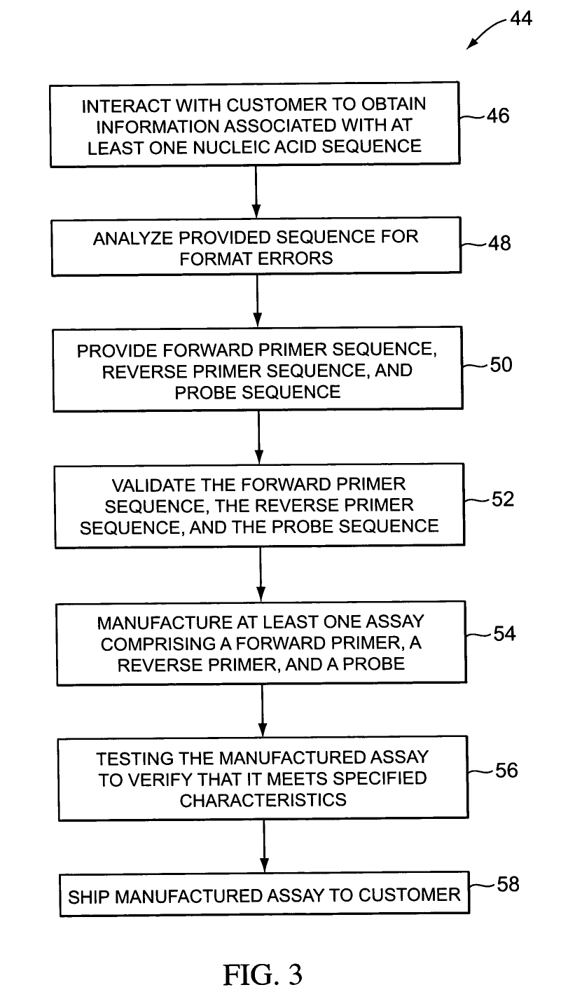

Referring to FIGS. 2 and 3, various configurations of the present disclosure can perform a method 44 for distributing a biotechnology product to a consumer. More particularly, the method can include utilizing a computer network 24 to interact at 46 with a consumer 28 to obtain information associated with (i.e., indicative of) at least one nucleic acid sequence. The target nucleic acid sequence obtained from the consumer can be, for example, a target RNA or DNA sequence, which itself can include an exon or a portion thereof, and/or a single nucleotide polymorphism (SNP). The information can further include information associated with a SNP location and/or an exon location. The provided nucleic acid sequence can be analyzed at 48 for format errors. If errors are detected, further interaction at 46 can be performed to correct the format errors.

Upon obtaining information from consumer 28, various methods of the present disclosure can provide, at 50, a forward primer sequence, a reverse primer sequence, and a probe sequence having specified characteristics. The forward primer sequence and the reverse primer sequence together can define an amplicon sequence, which lies within the target nucleic acid sequence. The probe sequence can be complementary to a portion of the amplicon sequence. Next, in various configurations, at least one of the forward primer sequence, the reverse primer sequence, and the probe sequence can be validated at 52, using, for example, a genome database such as database 40. Validation can include BLASTing of at least one of the sequences. At least one assay can be manufactured at 54. The manufactured assay can comprise a forward primer in accordance with the forward primer sequence, a reverse primer in accordance with the reverse primer sequence, and a probe in accordance with the probe sequence. In some configurations, the forward primer sequence, the reverse primer sequence, and/or the probe sequence can be a validated sequence from 52. The assay can be shipped at 58 to consumer 28.

Some configurations can test, at 56, the manufactured forward primer, the manufactured reverse primer, and/or the manufactured probe before delivery to verify that the assay meets specified characteristics. Tests at 56 can include, for example, performing mass spectroscopy on the manufactured assay to determine that an oligonucleotide sequence is correct, and/or performing a functional test to determine that an amplification has occurred and at least one allelic discrimination can be confirmed.

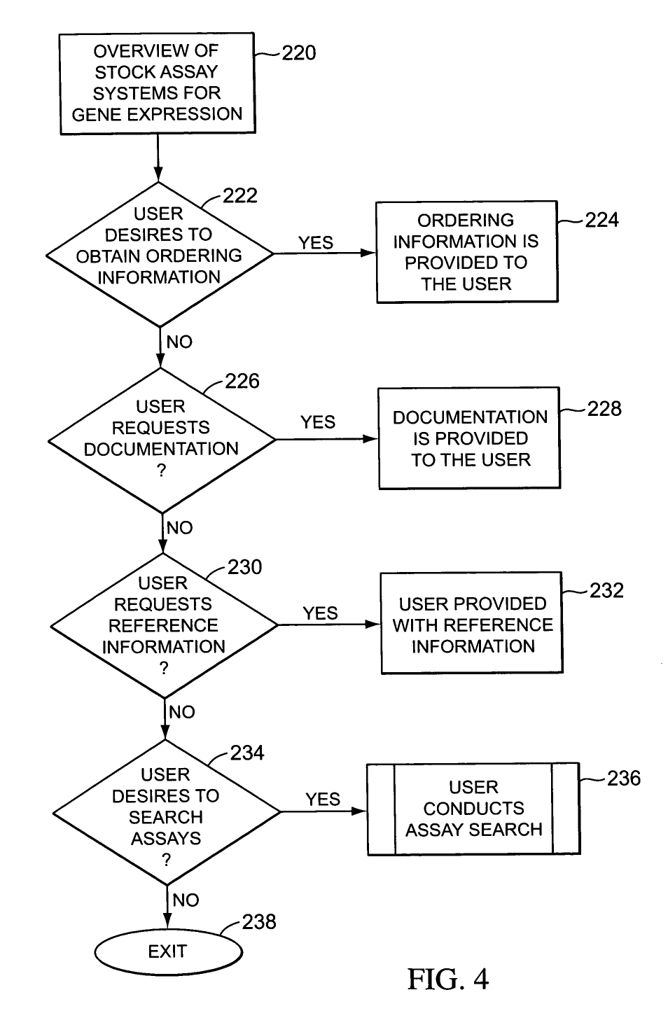

Referring to FIG. 4, if, after viewing the overview of stock assay systems at 220, the user desires to obtain ordering information regarding stock assays for gene expression as indicated by block 222, ordering information can then be provided to the user at block 224. In this regard, the user can be provided with information regarding the contents of the assay which will be provided as well as technical information regarding the assay. In addition, information regarding the volume and reactions to produce can be provided as well as the necessary instrument platform. Part number information can also be available for the assay, as well as part numbers for related equipment. In one non-limiting example, the user can be informed of the components of the gene expression assays which will be received by the user.

The user can also request documentation from the system as indicated in FIG. 4 at decision block 226. If at block 226 the user requests documentation regarding gene expression assays, the system delivers documentation regarding the stock assay at block 228. This information can be brochures, product bulletins, user bulletins as well as other type of instructional or other information. This information can be delivered either via download, fax, e-mail or hard copy. In some configurations, the user can select for delivery any number of the listed documents in any or all of the available formats for delivery.

Further, the user can request reference information at decision block 230. If the user requests reference information at decision block 230, the user can be provided at block 232 with reference information which can be links to publicly available databases. For example, the user at block 232 can be linked to the NCBI Reference Sequence Project (RefSeq) database. It is to be understood, however, that other suitable database can be referenced.

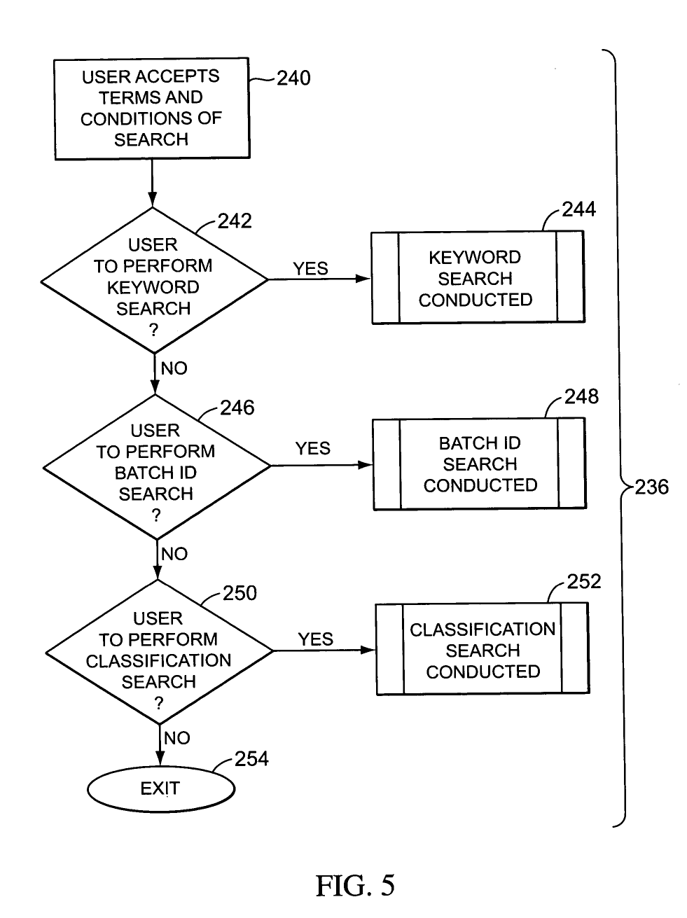

The user can decide to search gene expression assays as. represented by block 230. If the user decides to search gene expression assays at block 234, the user can be requested to accept certain terms and conditions of use for the assay search at block 240 (see FIG. 5). In addition to providing terms and conditions of use, the user can also be requested to provide information concerning the user such as name, institution, e-mail, phone number and/or address. In addition, the user can also be asked at block 240 whether the user would like information regarding products or services.

If the user accepts the terms and conditions of use, the user can be directed at block 242 to a window pane which allows the user to search for stock assays. The user can then be given the opportunity to search for gene expression assays by various techniques. For example, the user can at decision block 242 use keyword searching to find assays by searching for keywords such as gene name, gene symbol or gene ontology classification. If the user selects a keyword search at decision block 242, a keyword search can be conducted at block 244 as more fully disclosed below. The user can also decide to conduct a batch ID search at block 246 so as to find assays by searching for multiple accession numbers from public or private sources such as, for example, from Celera, Applied Biosystems or public databases. If the user selects to perform a batch ID search at block 246, a batch ID search can be performed at block 248 as will be more fully disclosed below. Finally, the user can decide to perform a classification search at decision block 250 to find assays by a suitable classification system such as the Celera Panther protein classification system.

If the user selects to perform a keyword search at block 242, the user can perform either a basic or an advanced keyword search. If a basic keyword is to be performed, the user is able to select the search field in which the search is to be conducted, as well as enter a specific search term. The specific fields which can be searched include the non-limiting examples: Gene Symbol, Gene Name, RefSeq Accession, Panther Function, Panther Process, GO Function, GO Process, GO ID, AB Assay ID, Celera gene (CG), Celera transcript (CT), Celera protein (CP), LocusLink ID, GenBank Nucleotide ID, GenBank Protein ID, Species, Chromosome, Cytoband, and RefSeq GI. If an advance keyword search is selected by the user, the user can insert search criteria for all of the fields described above.

If the user determines that it is desirable to conduct a batch ID search at block 246, a batch ID search can be conducted at block 248. The batch ID search can find assays by using a list identification numbers. In this regard, the user is able to search by identification numbers from a variety of sources such as: RefSeq accession number, GenBank Protein (GenPept) accession number, GenBank GI number, LocusLink, LocusLink gene symbol, Celera Gene (CG), Celera Transcript (CT), Celera Protein (CP), and AB Assay ID.

Finally, the user can decide at block 250 whether a classification search, such as using the Celera panther classification system, is to be conducted. The Celera Panther classification system is a system for classifying and predicting the functions of proteins in the context of sequence-relationships (see for example, U.S. patent application Ser. No. 11/735,606, filed Dec. 12, 2003, entitled “Methods for identifying, viewing, and analyzing syntenic and orthologous genomic regions between two or more species,” which is hereby incorporated by reference in its entirety). Assays can be assigned to a Panther category based upon a match to equivalently assigned Celera gene data. The Panther categories can be constructed up to three levels deep with assay assignments at any one of the three levels.

If the user desires to perform a classification search at block 250, a classification search can be conducted at block 252. The user is then able to search by molecular function categories involving a property of the protein or of a particular biochemical reaction performed by a protein, such as receptor, kinase or hydrolase. In addition, the user can search by biological process categories involving the biochemical reactions that work together towards a common biological objective. The process can be at the cellular level, such as glycolysis and signal transduction, or at the system level, such as immunity and defense, in sensory perception.

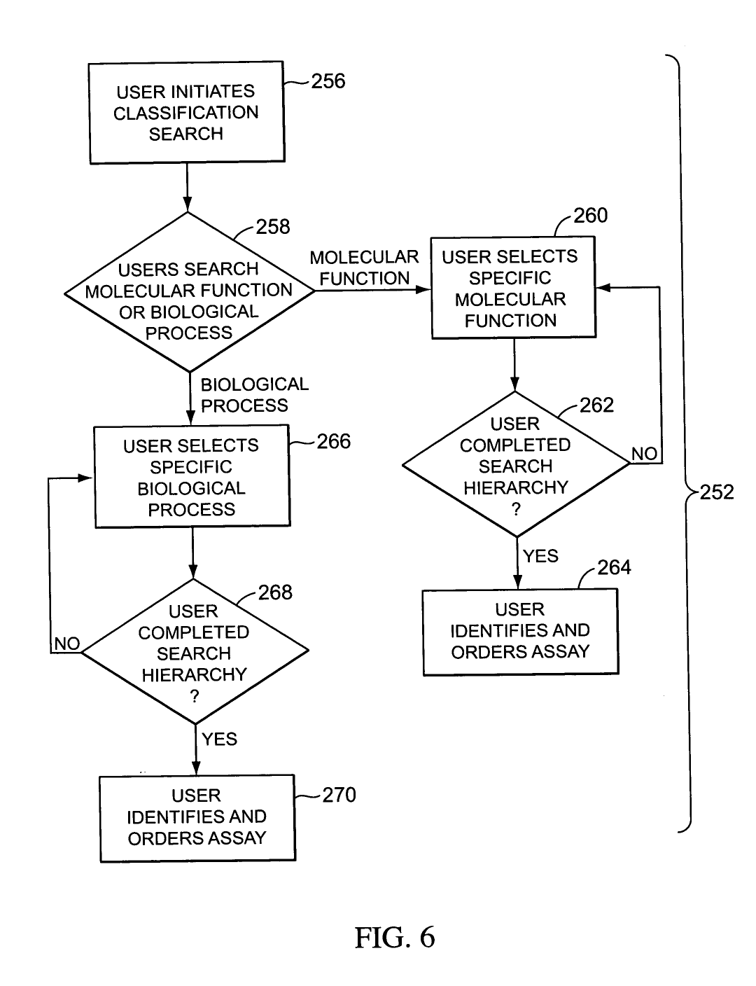

An example of the manner in which a classification search at block 252 can be conducted is shown in FIG. 6. In this regard, the user can initiate a classification search at block 256. After the user initiates the classification search, the user can decide whether the classification is to be conducted with respect to molecular function or biological process at block 258. If the user decides at block 258 to search by molecular function, then the user can review a hierarchy of molecular functions until a set of assays can be presented to the user for the desired molecular function. In this regard, the user can select at block 260 a category of molecular functions. The processing then proceeds to block 262 to allow the user to decide whether the hierarchy search has been completed. This can occur if there are no further subclassifications within the category searched. For example, if the category of molecular function which is selected is “receptor”, there can be seven categories associated with this molecular function, including three subcategories (i.e., protein kinase receptor, cytokine receptor and ligand-gated ion channel receptor). If at block 262, the user has decided to search a subcategory of molecular function, the user can then select another specific molecular function at block 260. For example, if the user selects the subcategory of “protein kinase receptor” a window pane can be displayed indicating the categories of protein kinase receptors. When the user has completed the hierarchal search at block 262, the user can then identify and order the assay at block 264.

Similarly, the user can select at block 258 to conduct a search based on biological processes. If the user makes the selection, the user selects one of a number of broad categories of biological processes which the system provides to the user at block 266. After selecting one of the broad categories of biological process at block 266, the user can determine whether the search hierarchy has been completed at block 268. If the user has not completed the search hierarchy (i.e., the relevant biological process displayed to the user contains subcategories), the user then again selects one of the subcategories at block 266. If the user has completed this search hierarchy at block 268, the user then identifies and orders the assay at 270.

A container can comprise assay reagents and components necessary to conduct PCR, with the exception of a target polynucleotide. The target polynucleotide can be provided by a user and mixed with the assay reagents or the target polynucleotide can be provided to the user in a second container to be mixed with the assay reagents.

The container can be a tube, vial, jar, capsule, ampule, or like vessel. The tube can have a removable cap and/or a replaceable cap. The cap can maintain the container sealed such that the container is water-tight and air-tight. The container can be hermetically sealed. The container can be open at a first end and closed at a second end. According to an embodiment, the first end can be tapered, for example, along the length of the container. The container can hold a mixture including assay reagents and have a volume of at least about 5 L, for example about 10 L or less. The maximum volume of the container can be about 25 L or less, and according to other embodiments, the volume can be greater than about 25 L.

The assay reagents in the container can contain a volume of assay reagents for more than one respective assay. For example, the assay reagents in the container can be divided and transferred into five respective reaction wells, for example, to conduct five identical and/or different assays. For another example, the assay reagents. in the container can be removed from the container and aliquoted into ten respective reaction wells. According to various embodiments, the container can contain a sufficient volume of assay reagents to complete at least 1, at least 5, at least 10, or at least 25 assays.

The container can contain at least one probe reactive with a target polynucleotide, wherein the probe can include a polynucleotide, a marker compound, for example, a marker dye, a quenchable dye, or a fluorescent reporter dye, a non-fluorescent quencher, a minor groove binder, or a combination thereof.

The probe can include a reporter dye such as VIC or 6-FAM linked to the 5′ end of the polynucleotide. VIC and 6-FAM dye-labeled probes are available from Applied Biosystems, Foster City, Calif. The minor groove binder can increase the melting temperature Tₘ without increasing the length of the polynucleotide. This can result in greater differences in Tₘ values between matched and mismatched probes that therefore can enable more accurate allelic discrimination. The probe can include a quencher (e.g., a non-fluorescent quencher) linked to the 3′ end of the polynucleotide. The quencher can inhibit fluorescence that can facilitate greater discrimination of reporter dye fluorescence.

The container can contain two different types of probes, wherein the polynucleotide and the reporter dyes differ. For example, the first type of probe can have a first polynucleotide with a VIC reporter dye attached to the 5′ end of the first polynucleotide and the second type of probe can have a second polynucleotide with a 6-FAM reporter dye attached to the 5′ end of the second polynucleotide and the first and second polynucleotides differ by at least one monomeric unit at the same location in the polynucleotide when the polynucleotides are aligned 5′ to 3′. The dye-labeled probes can be adapted to perform a heterozygous assay or a homozygous assay.

The probe can anneal to a complementary sequence between the forward and reverse primer sites. At the time of annealing, the probe can be intact and the proximity of the reporter dye to the quencher can result in suppression of fluorescence of the reporter dye. A polymerase can cleave a reporter dye only when the probe has completely, mostly, or substantially hybridized to the target polynucleotide sequence. When the reporter dye is cleaved from the probe, the relative fluorescence of the reporter dye can increase. The increase in relative fluorescence can only occur if the amplified target polynucleotide sequence is complementary, mostly complementary, or substantially complementary to the probe. Therefore, the fluorescent signal generated by PCR amplification can indicate which alleles are present in a sample. Mismatches between a probe and a target polynucleotide sequence can reduce efficiency of probe hybridization and/or a polymerase can be more likely to displace a mismatched probe without cleaving it and therefore not produce a fluorescent signal. For example, if one of two possible reporter dyes fluoresce during an assay, then the presence of a homozygous gene is indicated. For further example, if both possible reporter dyes fluoresce during an assay, then the presence of a heterozygous gene is indicated.

The container can contain at least one primer, wherein the primer can comprise a sequence that is shorter than the target polynucleotide. The primer can comprise a polynucleotide and/or a minor groove binder. The primer can comprise a sequence that is complimentary to, or mostly complimentary to, the target polynucleotide. For example, the primer can be at least 90% homologous to a corresponding length of the target polynucleotide, at least 80% homologous to a corresponding length of the target polynucleotide, at least 70% homologous to a corresponding length of the target polynucleotide, or at least 50% homologous to a corresponding length of the target polynucleotide.

The container can contain a thermostable DNA polymerase, such as, for example, thermus aquaticus (Taq), and at least 4 embodiments of a deoxyribonucleic acid (e.g., adenosine, tyrosine, cytosine, and guanine). The polymerase can be, for example, AMPLITAQ GOLD, available from Applied Biosystems, Foster City, Calif. According to various embodiments, the container can comprise components of a fluorogenic 5′ nuclease assay or other assay reagents that utilize 5′ nuclease chemistry, for example, TAQMAN minor groove binder probes, available from Applied Biosystems, Foster City, Calif. Some or all of the above-listed components can be replaced by or used with commercially-available products, for example, buffers or AMPLITAQ GOLD PCR MASTER MIX (Applied Biosystems, Foster City, Calif.).

A multi-well plate can also be provided in the kit and can include, for example, 96 or 384 positions for container placement. A plate can be substantially rectangular with an optional integrated structural feature for plate orientation. A plate can have a plurality of wells. The assay kit can include a plate adapted to hold a plurality of containers or tubes. The plate can be of unitary construction. At least one container can be integrated into a single plate, and the plate can have a plurality of containers in physical contact with each other. For example, the plate can be of unitary construction and have 96 containers in the form of receptacles. For further example, the plate can be of unitary construction and have 384 containers.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that can vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” include plural referents unless expressly and unequivocally limited to one referent. Thus, for example, reference to “a sequence” includes two or more different sequences. As used herein, the term “include” and its grammatical variants are intended to be non-limiting, such that recitation of items in a list is not to the exclusion of other like items that can be substituted or added to the listed items.

While particular embodiments have been described, alternatives, modifications, variations, improvements, and substantial equivalents that are or can be presently unforeseen can arise to applicants or others skilled in the art. Accordingly, the appended claims as filed and as they can be amended are intended to embrace all such alternatives, modifications variations, improvements, and substantial equivalents. seemed reasonable to assume, on the grounds that they are functionally essential, these key regulatory units of the genome can be evolutionarily conserved relative to a flanking sequence. Thus cis-regulatory modules can be detected computationally by interspecific comparison of the sequence surrounding the gene of interest, recognized as a block of sequence which has remained relatively similar between, for example, two species, excised by PCR and incorporated in an expression vector, and their function then studied by direct gene transfer methods.

The appropriate evolutionary species distance must be chosen; that is, not so close that unselected (i.e., “background”) sequence has not had time to diverge, but not so far that the pattern of conservation has been lost by too much divergence. But at the “right” distance, cis-regulatory modules stand out from the immediately flanking background as patches of well conserved sequence, usually several hundred base pairs in length, terminated at their boundaries by abrupt transitions to sequence that has diverged too greatly for easy computational alignment.

The use of interspecific sequence comparison to locate CRMs can decrease the percentage of false positives associated with using known algorithms to find TFBSs.

### SUMMARY OF THE DISCLOSURE

In accordance with the disclosure, there is provided a process for identifying a cis-regulatory module comprising aligning a target sequence with at least one sequence from a moderately distant species; determining a non-coding region of the target sequence, wherein the non-coding region comprises at least one of a high

What is claimed is:

1. A process for identifying a cis-regulatory module comprising:
aligning a target sequence with at least one sequence from a moderately distant species;
determining a non-coding region of the target sequence, wherein the non-coding region comprises at least one of a high level of conservation and suppression of indels; and
identifying at least one cis-regulatory module in the target sequence.

2. The process of claim 1, wherein the highly conserved region is identified by using a computer program.

3. The process of claim 2, wherein the computer program is BLASTn.

4. The process of claim 1, wherein at least 30% of the nucleotides in the at least one sequence from the moderately distant species aligns with the target sequence.

5. A process for identifying a transcription factor binding site comprising:
aligning a target sequence with at least one sequence from moderately distant species,
determining a non-coding region of the target sequence, wherein the non-coding region comprises at least one of a high level of conservation and a suppression of indels,
identifying at least one cis-regulatory module in the target sequence, and
performing an algorithm to locate at least one transcription factor binding site within the at least one cis-regulatory module.

6. The process of claim 5, wherein the at least one cis-regulatory module comprises about 12 transcription factor binding sites.

7. A process for identifying a gene that is regulated by a transcription factor binding site comprising:
providing a first gene that encodes a first protein; and
identifying sequences that the first protein is known to bind to within a cis-regulatory module near a second gene.

8. A method for providing assays to a consumer comprising:
providing at least one web-based user interface selected from the group consisting of:
a) an interface configured to receive an order for at least one stock assay,
b) an interface configured to receive a request for design of at least one custom assay and an order for said custom assay, and
c) an interface configured for a consumer to perform at least one search for at least one information item chosen from cis-regulatory modules, transcription factor binding sites, genes whose proteins bind these cis-regulatory module-embedded transcription factor binding sites, and SNPs that occur in cis-regulatory module-embedded transcription factor binding sites; and
delivering to the consumer at least one assay chosen from a custom assay and a stock assay in response to the order.

9. The method of claim 8, wherein the information item is chosen from genomic and biomedical information from at least one public or private source.

10. The method of claim 9, wherein the information item is a gene identification item selected from the group consisting of gene symbol, gene name, RefSeq accession number, Panther function, Panther process, Panther family name, Panther subfamily name, Panther family ID, GO function, GO process, GO identifier, GO subcellular location, Applied Biosystems identifier, Celera gene identifier (CG), Celera transcript identifier (CT), Celera protein identifier (CP), LocusLink identifier, GenBank nucleotide identifier, GenBank protein identifier, species identifier, chromosome identifier, haplotype identifier, cytoband identifier, RefSeq GI identifier, and combinations thereof.

11. The method of claim 8, wherein the at least one search comprises a batch search.

12. The method of claim 8, wherein the at least one search comprises a gene classification search.

13. The method of claim 8, wherein the gene classification search comprises a biological process search.

14. The method of claim 8, wherein the gene classification search comprises a molecular function search.

15. The method of claim 8, wherein the gene classification search comprises a subcellular location search.
